(19) United States
(12) Patent Application Publication
(10) Pub. No.: US 2013/0079312 A1
(43) Pub. Date: Mar. 28, 2013

(54) Method of Treating Hair Loss Due to Systemic Chemotherapy

(71) Applicant: Joel E. Bernstein, Deerfield, IL (US)

(72) Inventor: Joel E. Bernstein, Deerfield, IL (US)

(73) Assignee: ELORAC, LTD., Vernon Hills, IL (US)

(21) Appl. No.: 13/247,125

(22) Filed: Sep. 28, 2011

(51) Int. Cl. A61K 31/4164; A61P 17/14; A61K 31/573

(52) U.S. Cl. 514/171; 514/401

(57) ABSTRACT

Methods and composition for preventing and/or treating hair loss associated with the administration of cancer chemotherapy using vasoconstrictors are disclosed. Optionally corticosteroids are also in the formulation.

### BACKGROUND

Methods and compositions using vasoconstrictors for preventing and/or treating hair loss associated with the administration of cancer chemotherapy, are disclosed.

One of the most distressing side effects of systemic cytotoxic chemotherapy in cancer patients is the loss of some or all of their hair (chemotherapy-induced alopecia). This complication of cancer treatment is especially distressing in children and women of any age. Currently, the principal cosmetic or esthetic solution to this problem is the use of real or artificial hair wigs. Attempts to ameliorate such hair loss by modifying chemotherapy regimens have not proved successful.

### SUMMARY

A method of preventing or reducing hair loss associated with cancer chemotherapy is disclosed. The method includes applying to a principal hair-bearing area of the body a formulation that includes locally effective vasoconstrictor compounds in vehicles suitable for topical application. Application may be just prior to and/or immediately after administration of the cancer chemotherapy.

A principal hair-bearing area includes scalp, eyebrows, axillae and combinations thereof.

Suitable, effective vasoconstrictor compounds include oxymetazoline, naphazoline, tetrahydrozoline, xylometazoline, salts and combinations thereof.

The topical formulations containing the vasoconstrictor compounds may continue to be applied to the hair-bearing area of the body one (1) to four (4) times daily for periods of several days to several weeks after completion of the chemotherapy course.

A locally active corticosteroid may be used with the vasoconstrictor compounds. The resulting formulation(s) are also applied to the selected hair-bearing areas of the body as provided herein.

The locally active corticosteroid may be fluorinated or non-fluorinated.

### DETAILED DESCRIPTION OF THE DISCLOSURE

Methods for preventing bodily hair loss due to systemic administration of cytotoxic agents for cancer chemotherapy. The inventor discovered, surprisingly, a method of preventing and/or treating chemotherapy-induced alopecia utilizing vehicles suitable for topical application to the scalp. The vehicles contain one or more of the closely related vasoconstrictor compounds, oxymetazoline, naphazoline, tetrahydrozoline and xylometazoline. By applying such formulations to the scalp (and, if desired, other hair-bearing areas of the body) of patients just prior to the administration of chemotherapy, and reapplying the formulation to the scalp 1-4 times daily thereafter, some or most of the hair loss can be avoided.

The inventor also discovered that the incorporation of a corticosteroid compound into the topical formulation along with a vasoconstrictor compound may further decrease hair loss while reducing possible local skin irritation from the vasoconstrictor compound. Such corticosteroids include all non-fluorinated corticosteroids such as hydrocortisone, methyl prednisolone and prednicarbate, or fluorinated corticosteroids such as triamcinolone, betamethasone and mometasone.

Methods are also disclosed to treat or ameliorate hair loss in patients who have already received some chemotherapy and have already lost some scalp hair.

Solutions, creams, gels, foams, ointments, lotions and suspensions suitable for application to the scalp or other hair-bearing areas of the body are prepared employing about 0.01 to about 5% by weight of a vasoconstrictor such as oxymetazoline, naphazoline, tetrahydrozoline or xylometazoline. Such formulations may be applied to hair-bearing areas from 5 to 60 minutes prior to the administration of chemotherapy to patients, and then continue to be applied from one to four times daily for the duration of the course of chemotherapy. Formulations disclosed herein or their equivalents may be applied for several days to several weeks after the completion of the chemotherapy course.

Alternatively, such formulations may be applied to the scalp and other hair-bearing areas within 1 to 60 minutes after administration of chemotherapy to patients and then reapplied to the scalp one to four times daily for from 1 to 7 days following each day chemotherapy is administered.

Additionally, a locally active corticosteroid can be incorporated into the above mentioned formulations containing a vasoconstrictor, and the resulting formulations can be applied in a fashion identical to or similar to that described above for topical formulations containing only the vasoconstrictor.

### MATERIALS AND METHODS

### Example 1

A cream is prepared by incorporating 0.05% oxymetazoline in a pharmaceutically acceptable cream vehicle and applied to the scalp of cancer patients 30 minutes prior to the intravenous administration of busulfan. This cream is then applied twice daily between courses of chemotherapy to reduce hair loss.

### Example 2

Oxymetazoline 2.0% by weight is incorporated into a hydroethanolic foam vehicle and the foam applied to the scalp and eyebrows sixty (60) minutes prior to intravenous infusion of nitrogen mustard. The foam is then applied to the scalp and eyebrows three times daily for two week intervals between recurring administrations of intravenous chemotherapy with a resulting prevention of major hair loss.

### Example 3

Tetrahydrozoline 5.0% by weight is incorporated into a solution of 2.5% hydrocortisone by weight and the resulting formulation applied to the scalp, eyebrows and axillae immediately after the intravenous administration of melphalan hydrochloride and applications of the tetrahydrozoline/hydrocortisone solution continued four times daily for three (3) days following each course of melphalan chemotherapy. Patients who receive such a therapeutic regimen will experience much less hair loss than patients receiving melphalan without the preventative hair loss regimen.

### Example 4

Patients receiving intravenous injections of docetaxel for breast cancer have a scalp lotion containing 0.01% naphazoline applied to their scalps thirty (30) minutes prior to and fifteen (15) minutes after administration of each course of intravenous docetaxel. Most such patients lose little, if any, of their hair.

### Example 5

A gel containing 0.5% oxymetazoline and 0.1% triamcinolone acetonide is applied to the scalp and eyebrows of patients thirty (30) minutes before and thirty (30) minutes after receiving epirubicin hydrochloride injections for axillary node tumor involvement following resection of primary breast cancer. The gel is then applied twice daily to the scalp and eyebrows for one week following the injection of epirubicin. Patients receiving such scalp and eyebrow treatments lose much less hair than patients receiving epirubicin without such scalp treatments.

1. A method of preventing or reducing hair loss associated with cancer chemotherapy, the method comprising applying to a principal hair-bearing area of the body a formulation comprising locally effective vasoconstrictor compounds in vehicles suitable for topical application just prior to and/or immediately after administration of the cancer chemotherapy.

2. The method of claim 1, wherein the topical formulations comprising the vasoconstrictor compounds continue to be applied to the hair-bearing area of the body one (1) to four (4) times daily for periods of several days to several weeks after completion of the chemotherapy course.

3. The method of claim 1, wherein the effective vasoconstrictor compounds are selected from the group consisting of oxymetazoline, naphazoline, tetrahydrozoline, xylometazoline, salts and combinations thereof.

4. The method of claim 1, further comprising a locally active corticosteroid with the vasoconstrictor compound, and wherein the resulting formulation(s) are applied to the selected hair-bearing areas of the body as provided in claims 1 and 2.

5. The method of claim 4, wherein the locally active corticosteroid may be fluorinated or non-fluorinated.

6. The method of claim 1, wherein a hair-bearing area is selected from the group consisting of scalp, eyebrows, axillae and combinations thereof.
